(19) United States
(12) Patent Application Publication
(10) Pub. No.: US 2003/0060640 A1
(43) Pub. Date: Mar. 27, 2003

(54) Method for the preparation of 5-cyano-1-(4-fluorophenyl)-1,3-dihydroisobenzofurans

(75) Inventor: Hans Petersen, Vanlose (DK)

Correspondence Address: DARBY & DARBY P.C., Post Office Box 5257, New York, NY, 10150-5257, US

(73) Assignee: H. Lundbeck A/S, Valby-Copenhagen (DK)

(21) Appl. No.: 10/233,132

(22) Filed: Aug. 30, 2002

Related U.S. Application Data
(63) Continuation of application No. PCT/DK01/00186, filed on Mar. 16, 2000.

(30) Foreign Application Priority Data
Mar. 16, 2000 (DK) PA 2000 00437

(51) Int. Cl. C07D 307/93; C07D 307/87

(52) U.S. Cl. 549/462

(57) ABSTRACT

A method for the preparation of 5-cyano-1-(4-fluorophenyl)-1,3-dihydroisobenzo-furan comprising conversion of a 5-substituted 1-(4-fluorophenyl)-1,3-dihydro-isobenzofuran derivative.

This application is a continuation of International Application No. PCT/DK01/00186, filed Mar. 16, 2001. The prior application is hereby incorporated by reference, in its entirety.

The present invention relates to a method for the preparation of 5-cyano-1-(4-fluorophenyl)-1,3-dihydroisobenzofuran which is an intermediate used for the manufacture of the well-known antidepressant drug citalopram, 1-[3-(dimethylamino)propyl]-1-(4-fluorophenyl)-1,3-dihydro-5-isobenzofurancarbonitrile.

### BACKGROUND OF THE INVENTION

Citalopram is a well-known antidepressant drug that has now been on the market for some years and has the following structure:

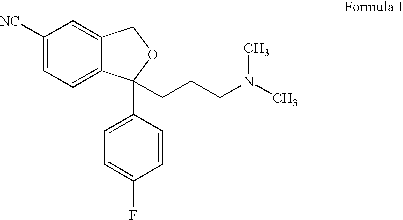

It is a selective, centrally active serotonin (5-hydroxytryptamine; 5-HT) reuptake inhibitor, accordingly having antidepressant activities. The antidepressant activity of the compound has been reported in several publications, eg. J. Hyttel Prog. Neuro-Psychopharmacol. & Biol. Psychiat. 1982, 6, 277-295 and A. Gravem Acta Psychiatr. Scand. 1987, 75, 478-486. The compound has further been disclosed to show effects in the treatment of dementia and cerebrovascular disorders, EP-A-474580.

Citalopram was first disclosed in DE 2,657,013, corresponding to U.S. Pat. No. 4,136,193. This patent publication describes the preparation of citalopram by one method and outlines a further method, which may be used for preparing citalopram.

According to the process described, the corresponding 1-(4-fluorophenyl)-1,3-dihydro-5-isobenzofurancarbonitrile is reacted with 3-(N,N-dimethylamino)propyl-chloride in the presence of methylsulfinylmethide as condensing agent. The starting material was prepared from the corresponding 5-bromo derivative by reaction with cuprous cyanide.

International patent application No. WO 98/019511 discloses a process for the manufacture of citalopram wherein a (4-(cyano, alkyloxycarbonyl or alkylaminocarbonyl)-2-hydroxymethylphenyl-(4-fluorophenyl)methanol compound is subjected to ring closure. The resulting 5-( alkyloxycarbonyl or alkylaminocarbonyl)-1-(4-fluorophenyl)-1,3-dihydroisobenzofuran is converted to the corresponding 5-cyano derivative and the 5-cyano derivative is then alkylated with a (3-dimethylamino)propylhalogenide in order to obtain citalopram.

It has now, surprisingly, been found that citalopram may be manufactured by a novel favourable process where a 5-substituted 1-(4-fluorophenyl)-1,3-dihydroisobenzofuran is converted to the corresponding 5-cyano-1-(4-fluorophenyl)-1,3-dihydroisobenzofuran before being alkylated by a 3-dimethylaminopropyl-group.

### DESCRIPTION OF THE INVENTION

Accordingly, the present invention relates to a novel method for the preparation of an intermediate in the preparation of citalopram having the formula

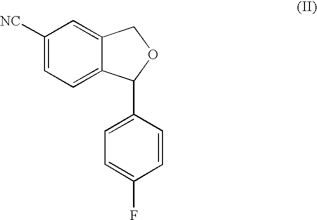

by conversion of a compound of formula

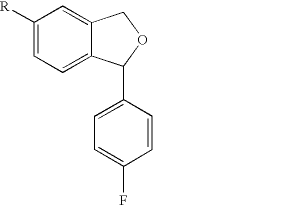

wherein R is halogen, a group of the formula CF₃—(CF₂)ₙ—SO₂—O—, wherein n is 0-8, —OH, —CHO, —CH₂OH, CH₂NH₂, —CH₂NO₂, —CH₂Cl, —CH₂Br, —CH₃, —NHR¹, —COOR², —CONR²R³ wherein R² and R³ are selected from hydrogen optionally substituted alkyl, aralkyl or aryl and R¹ is hydrogen or alkylcarbonyl, or a group of formula

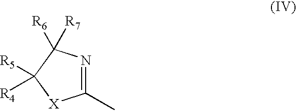

where in

X is O or S;

R⁴—R⁵ are each independently selected from hydrogen and C₁₋₆ alkyl or R⁴ and R⁵ together form a C₂₋₅ alkylene chain thereby forming a spiro ring; R⁶ is selected from hydrogen and C₁₋₆ alkyl, R⁷ is selected from hydrogen, C₁₋₆ alkyl, a carboxy group or a precursor group therefore, or R⁶ and R⁷ together form a C₂₋₅ alkylene chain thereby forming a spiro ring.

This intermediate product of formula (II) may be converted to citalopram by alkylation as described above.

In another aspect, the present invention relates to an antidepressant pharmaceutical composition comprising citalopram manufactured by the process of the invention.

According to one embodiment of the invention, wherein R is halogen, and the compound of formula (III) is converted to a compound of formula (II) by a reaction with a cyanide source optionally in the presence of a catalyst.

According to a further embodiment of the invention, wherein R is a triflate group of the formula CF₃—(CF₂)ₙ—SO₂—O—, wherein n is 0, 1, 2, 3, 4, 5, 6, 7 or 8, the compound of formula (III) is converted to a compound of formula (II) by reaction with a cyanide source optionally in the presence of a catalyst.

The cyano sources may conveniently be selected from a group consisting of cyanide sources such as NaCN, KCN, Zn(CN)₂, Cu(CN) or (R″)₄NCN wherein each R″ represents C₁₋₈-alkyl or optionally two R″ together with the nitrogen form a ring structure or combinations thereof.

The cyanide source is used in a stoichiometric amount or in excess, preferably 1-2 equivalents are used per equivalent starting material.

When R is halogen or a group of the formula CF₃—(CF₂)ₙ—SO₂—O—, wherein n is 0-8, the reaction of the present invention is performed in the presence or absence of a catalyst. The catalysts are i.e., Ni (0), Pd(0) or Pd(II) catalysts as described by Sakakibara et. al. in Bull. Chem. Soc. Jpn. 1988, 61, 1985-1990. Preferred catalysts are Ni(PPh₃)₃, Pd(PPh₃)₄, Ni(PPh)₂Cl or Pd(PPh)₂Cl₂.

In a particularly preferred embodiment, a Nickel(0) complex is prepared in situ before the cyanide exchange reaction by reduction of a Nickel(II) precursor such as NiCl₂ or NiBr₂ by a metal, such as zinc, magnesium or manganese in the presence of excess of complex ligands, preferably triphenylphosphine.

The Pd or Ni-catalyst is conveniently used in an amount of 0.5-10, preferably 2-5 mol %.

In one embodiment of the invention, the reaction is carried out in the presence of a catalytic amount of Cu⁺ or Zn²⁺.

Catalytic amounts of Cu⁺ and Zn²⁺, respectively, means substoichiometric amounts such as 0.1-5, preferably 1-3%. Conveniently, about ½ eq. is used per eq. Pd.

Any convenient source of Cu⁺ and Zn⁺⁺ may be used. Cu⁺ is preferably used in the form of CuI and Zn²⁺ is conveniently used as the Zn(CN)₂ salt.

The reactions may be performed in any convenient solvent as described in Sakakibara et. al. in Bull. Chem. Soc. Jpn. 1988, 61, 1985-1990. Preferred solvents are acetonitrile, ethylacetate, TUF, DMF or NMP.

In one aspect of the invention, a compound of Formula IV wherein R is Cl is reacted with NaCN in the presence of a Ni(PPh₃)₃ which is preferably prepared in situ as described above.

In another aspect of the invention, a compound of formula IV, wherein R is Br or I, is reacted with KCN, NaCN, CuCN or Zn(CN)₂ in the presence of Pd(PPh₃)₄. In a particular aspect of the invention, substoichiometric amounts of Cu(CN) and Zn(CN)₂ are added as recycleable cyanide sources .

In another aspect of the invention, a compound of formula IV, wherein R is Br or I, is converted to the corresponding cyano compound by reaction with Cu(CN) without catalyst. In a preferred embodiment, the reaction is performed at elevated temperature.

In a particular aspect of this invention, the cyanide exchange reaction is performed as a neat reaction i.e. without added solvent.

In another aspect of the invention, the cyanide exchange reaction is performed in an ionic liquid of the general formula (R′)₄N⁺, X⁻, wherein R′ are alkyl-groups or two of the R′ groups together form a ring and X⁻ is the counterion. In one embodiment of the invention, (R′)₄N⁺X⁻ represents

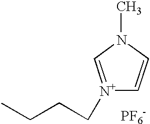

In another particular aspect of this invention, the cyanide exchange reaction is conducted with apolar solvents such as benzene, xylene or mesitylene and under the influence of microwaves by using i.e. Synthewave 1000™ by Prolabo. In a particular aspect of this invention, the reaction is performed without added solvent.

The temperature ranges are dependent upon the reaction type. If no catalyst is present, preferred temperatures are in the range of 100-200° C. However, when the reaction is conducted under the influence of microwaves, the temperature in the reaction mixture may raise to above 300° C. More preferred temperature ranges are between 120-170° C. The most preferred range is 130-150° C.

If catalyst is present, the preferred temperature range is between 0 and 100° C. More preferred are temperature ranges of 40-90° C. Most preferred temperature ranges are between 60-90° C.

Other reaction conditions, solvents, etc. are conventional conditions for such reactions and may easily be determined by a person skilled in the art.

In another embodiment of the invention, wherein R is an oxazoline or a thiazoline group of formula

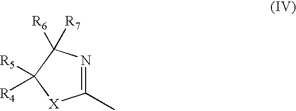

wherein X, R⁴, R⁵, R⁶ and R⁷ are as defined above, the conversion to a cyano group may be carried out with a dehydration agent or alternatively, where X is S, by thermal cleavage of the thiazoline ring or treatment with a radical initiator, such as peroxide or with light.

The dehydration agent may be any suitable dehydration agent conventionally used in the art, such as phosphoroxytrichloride, thionylchloride, phosphorpentachloride, PPA (polyphosphoric acid) and P₄O₁₀. The reaction may be carried out in the presence of an organic base, such as pyridine or a catalytic amount of a tertiary amide.

Preferably, the oxazoline or thiazoline derivative of formula (IV) is treated with SOCl₂ as a dehydrating agent and the reaction is carried out in toluene comprising a catalytic amount of N,N-dimethylformamide.

Alternatively, the dehydration agent may be a Vilsmeier reagent, i.e. a compound which is formed by reaction of a chlorinating agent, preferably an acid chloride, e.g. phosgene, oxalyl chloride, thionyl chloride, phosphoroxychloride, phosphorpentachloride, trichloromethyl chloroformate, also briefly referred to as “diphosgene”, or bis(trichloromethyl) carbonate, also briefly referred to as “triphosgene”, with a tertiary amide such as N,N-dimethylformamide or a N,N-dialkylalkanamide, e.g N,N-dimethylacetamide. A classic Vilsmeier reagent is the chloromethylenedimethyliminium chloride. The Vilsmeier reagent is preferably prepared in situ by adding the chlorinating agent to a mixture containing the starting oxazoline or thiazoline derivative of formula (IV) and the tertiary amide.

When X is S, the conversion of the thiazoline group of formula (IV) into the cyano group is made by thermal transformation, the thermal decomposition of the thiazoline group is preferably carried out in an anhydrous organic solvent, more preferably an aprotic polar solvent, such as N,N-dimethylformamide, N,N-dimethylacetamide, dimethylsulfoxide or acetonitrile. The temperature at which the thermal decomposition transforms the 2-thiazolyl group to a cyano group is between 60° C. and 140° C. The thermal decomposition may conveniently be carried out by reflux in a suitable solvent, preferably acetonitrile. The thermal cleavage may conveniently be carried out in the presence of oxygen or an oxidation agent. A thiazoline group of formula (IV) where X is S and R⁷ is a carboxy group or a precursor for a carboxy group can also be converted to a cyano group by treatment with a radical initiator such as light or peroxides.

According to a further embodiment of the invention, wherein R is a formaldehyde group, the compound of formula (III) is converted to a compound of formula (II) by conversion of the aldehyde group to an oxime followed by dehydration of the oxime group.

The conversion of the formyl group to a cyano group may thus be carried out by reaction with a reagent R⁸—V—NH₂ wherein R⁸ is hydrogen, lower alkyl, aryl or heteroaryl and V is O, N or S, followed by dehydration with a common dehydrating agent, for example thionylchloride, acetic anhydride/pyridine, pyridine/HCl or phosphor pentachloride. Preferred reagents R⁸—V—NH₂ are hydroxylamine and compounds wherein R⁸ is alkyl or aryl and V is N or O.

According to a further embodiment of the invention, wherein R is a —COOH group, the compound of formula (III) is converted to a compound of formula (II) by conversion to the amide via the corresponding acid chloride or an ester thereof followed by dehydration of the amide.

The acid chloride is conveniently obtained by treatment of the acid with POCl₃, PCl₅ or SOCl₂ neat or in a suitable solvent, such as toluene or toluene comprising a catalytic amount of N,N-dimethylformamide. The ester is obtained by treatment of the carboxylic acid with an alcohol, in the presence of an acid, preferably a mineral acid or a Lewis acid, such as HCl, H₂SO₄, POCl₃, PCl₅ or SOCl₂. Alternatively, the ester may be obtained from the acid chloride by reaction with an alcohol. The ester or the acid chloride is then converted to an amide by amidation with ammonia or a C₁₋₆ alkylamine, preferably t-butyl amine.

The conversion to amide may also be obtained by reaction of the ester with ammonia or an alkylamine under pressure and heating.

The amide group is then converted to a cyano group by dehydration. The dehydrating agent may be any suitable dehydrating agent, and the optimal agent may easily be determined by a person skilled in the art. Examples of suitable dehydrating agents are SOCl₂, POCl₃ and PCl₅, preferably SOCl₂.

In a particularly preferred embodiment, the carboxylic acid is reacted with an alcohol, preferably ethanol, in the presence of POCl₃, in order to obtain the corresponding ester, which is then reacted with ammonia thereby giving the corresponding amide, which in turn is reacted with SOCl₂ in toluene comprising a catalytic amount of N,N-dimethylformamide.

Alternatively, a compound where R is —COOH may be reacted with chlorosulfonyl isocyanate in order to form the nitrile, or treated with a dehydrating agent and a sulfonamide as described in WO 00/44738.

Thus, a compound of formula (III) wherein R is a —COOR² group may be converted to a compound of formula (II) by conversion to the amide followed by dehydration.

Further, a compound of formula (III) wherein R is a —CONR²R³ group may be converted to a compound of formula (II) by dehydration to form the cyano group.

In another embodiment of the invention, wherein R is a —NHR¹ group, the compound of formula (III) is converted to a compound of formula (II) by hydrolysation to form a free amino group followed by diazotation of the free amino group and reaction with a cyanide source.

The cyanide source used is most preferably NaNO₂, CuCN and/or NaCN. When R′ is C₁₋₆ alkylcarbonyl, it is initially subjected to hydrolysis thereby obtaining the corresponding compound wherein R¹ is H which is then converted as described above. The hydrolysis may be performed either in acidic or basic environment.

Compounds of formula (III) wherein R is a —CH₂NO₂ group may be converted to a compound of formula (II) by treatment with TMSI to form the cyano group.

Compounds of formula (III) wherein R is a —CH₂NH₂ group may be converted to a compound of formula (II) by oxidation in presence of Copper(I)chloride to form the cyano group.

Compounds of formula (III) wherein R is a —CH₂Cl group may be converted to a compound of formula (II) by reaction with AgNO₂ to form the corresponding —CH₂NO₂ group and followed by a treatment with TMSI to form the cyano group.

Compounds of formula (III) wherein R is a —CH₂Br group, may be converted to a compound of formula (II) by reaction with AgNO₂ to form the corresponding —CH₂NO₂ group and followed by a treatment with TMSI to form the cyano group; or a treatment with NH₃ to form the corresponding —CH₂NH₂ group and followed by an oxidation in presence of Copper(I)chloride to form the cyano group.

Compounds of formula (III) wherein R is a —CH₃ group may be converted to a compound of formula (II) by treatment with a base and secondly with R⁹ONO₂, wherein R⁹ is a C₁₋₆-alkyl, to form the corresponding —CH₂NO₂ group and followed by a treatment with TMSI to form the cyano group.

Compounds of formula (III) wherein R is a —CH₂OH group may be converted to a compound of formula (II) by treatment with SOCl₂ or SOBr₂ to form the corresponding —CH₂Cl group or —CH₂Br group followed by conversion to cyano as described above.

Starting material of Formula (III) wherein R is halogen may be prepared as described in GB 1526331, compounds of Formula IV wherein R is —O—SO₂—(CF₂)—CF₃ and —OH may be prepared analogous to the compounds described in WO 00/13648, compounds of Formula IV wherein R is an oxazoline or a thiazoline group may be prepared analogous to the compounds described in WO 00/23431, compounds of Formula IV wherein R is a CH₂OH group may be prepared analogous to the compounds described in PCT/DK/0100123, compounds of Formula IV wherein R is formaldehyde may be prepared analogously to the compounds described in WO 99/30548, compounds of Formula IV wherein R is —COOH, and esters and amides thereof may be prepared analogously to the compounds described in WO 98/19513 and compounds of Formula IV wherein R is —NHR¹ may be prepared analogously to the compounds described in WO 98/19512.

Citalopram is on the market as an antidepressant drug in the form of the racemate. However, in the near future the active S-enantiomer of citalopram is also going to be introduced to the market.

S-citalopram may be prepared by separation of the optically active isomers by chromatography.

Throughout the specification and claims, the term alkyl refers to a branched or unbranched alkyl group having from one to six carbon atoms inclusive, such as methyl, ethyl, 1-propyl, 2-propyl, 1-butyl, 2-butyl, 2-methyl-2-propyl, 2,2-dimethyl-1-ethyl and 2-methyl-1-propyl.

Similarly, alkenyl and alkynyl, respectively, designate such groups having from two to six carbon atoms, including one double bond and triple bond respectively, such as ethenyl, propenyl, butenyl, ethynyl, propynyl and butynyl.

The term aryl refers to a mono- or bicyclic carbocyclic aromatic group, such as phenyl and naphthyl, in particular phenyl.

The term aralkyl refers to aryl-alkyl, wherein aryl and alkyl is as defined above.

Halogen means chloro, bromo or iodo.

Citalopram may be used as the free base, in particular as the free base in crystalline form, or as a pharmaceutically acceptable acid addition salt thereof. As acid addition salts, such salts formed with organic or inorganic acids may be used. Exemplary of such organic salts are those with maleic, fumaric, benzoic, ascorbic, succinic, oxalic, bismethylenesalicylic, methanesulfonic, ethanedisulfonic, acetic, propionic, tartaric, salicylic, citric, gluconic, lactic, malic, mandelic, cinnamic, citraconic, aspartic, stearic, palmitic, itaconic, glycolic, p-aminobenzoic, glutamic, benzene sulfonic and theophylline acetic acids, as well as the 8-halotheophyllines, for example 8-bromotheophylline. Exemplary of such inorganic salts are those with hydrochloric, hydrobromic, sulfuric, sulfamic, phosphoric and nitric acids.

The acid addition salts of the compounds may be prepared by methods known in the art. The base is reacted with either the calculated amount of acid in a water miscible solvent, such as acetone or ethanol, with subsequent isolation of the salt by concentration and cooling, or with an excess of the acid in a water immiscible solvent, such as ethylether, ethylacetate or dichloromethane, with the salt separating spontaneously.

The pharmaceutical compositions of the invention may be administered in any suitable way and in any suitable form, for example orally in the form of tablets, capsules, powders or syrups, or parenterally in the form of usual sterile solutions for injection.

The pharmaceutical formulations of the invention may be prepared by conventional methods in the art. For example, tablets may be prepared by mixing the active ingredient with ordinary adjuvants and/or diluents and subsequently compressing the mixture in a conventional tabletting maschine. Examples of adjuvants or diluents comprise: Corn starch, potato starch, talcum, magnesium stearate, gelatine, lactose, gums, and the like. Any other adjuvant or additive, colourings, aroma, preservatives etc. may be used provided that they are compatible with the active ingredients.

Solutions for injections may be prepared by solving the active ingredient and possible additives in a part of the solvent for injection, preferably sterile water, adjusting the solution to the desired volume, sterilising the solution and filling it in suitable ampoules or vials. Any suitable additive conventionally used in the art may be added, such as tonicity agents, preservatives, antioxidants, etc.

1. A method for the preparation of 5-cyano-1-(4-fluorophenyl)-1,3-dihydroisobenzofuran 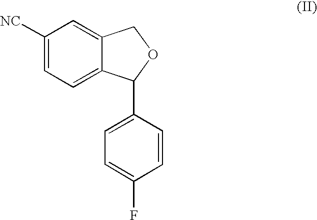
comprising converting a 5-substituted 1-(4-fluorophenyl)-1,3-dihydroisobenzo-furan derivative of the formula 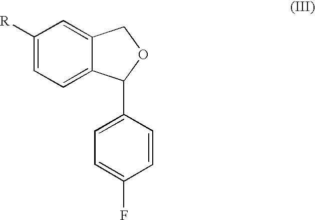
wherein R represents halogen, a group of the formula CF₃—(CF₂)ₙ—SO₂—O—, wherein n is 0-8, —OH, —CHO, —CH₂OH, —CH₂NH₂, —CH₂NO₂, —CH₂Cl, —CH₂Br, —CH₃, —NHR¹ —COOR², —CONR²R³ wherein R² and R³ are selected from hydrogen, optionally substituted alkyl, aralkyl or aryl, and R¹ is hydrogen or alkylcarbonyl, or a group of formula 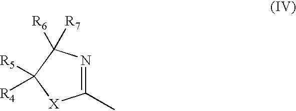
wherein
X is O or S;
R⁴ and R⁵ are each independently selected from hydrogen and C₁₋₆ alkyl, or R⁴ and R⁵ together form a C₂₋₅ alkylene chain thereby forming a spiro ring; R⁶ is selected from hydrogen and C₁₋₆ alkyl, R⁷ is selected from hydrogen, C₁₋₆ alkyl, a carboxy group or a precursor group therefore, or R⁶ and R⁷ together form a C₂₋₅ alkylene chain thereby forming a Spiro ring.

2. The method of claim 1, further comprising the step of alkylating the compound of formula (II) to form citalopram, and isolating citalopram or a pharmaceutically acceptable salt thereof.

3. The method of either of claims 1 or 2 wherein R is halogen, and the step of converting a compound of formula (III) to a compound of formula (II) comprises the step of reacting a compound of formula (III) with a cyanide source, optionally in the presence of a catalyst.

4. The method of either of claims 1 or 2 wherein R is a triflate group of the formula CF₃—(CF₂)ₙ—SO₂—O—, wherein n is 0, 1, 2, 3, 4, 5, 6, 7 or 8, and the step of converting a compound of formula (III) to a compound of formula (II) comprises reacting a compound of formula (III) with a cyanide source, optionally in presence of a catalyst.

5. The method of either of claims 1 or 2 wherein R is an oxazoline or a thiazoline group of formula (IV), and the step of converting a compound of formula (III) to a compound of formula (II) comprises treating a compound of formula (III) with a dehydrating agent, or alternatively where X is S, by thermal cleavage of the thiazoline ring or treatment with a radical initiator.

6. The method of either of claims 1 or 2 wherein R is a formaldehyde group, and the step of converting a compound of formula (III) to a compound of formula (II) comprises converting the aldehyde group to an oxime followed by dehydration of the oxime group.

7. The method of either of claims 1 or 2 wherein R is a —COOH group, and the step of converting a compound of formula (III) to a compound of formula (II) comprises converting the amide via the corresponding acid chloride or an ester thereof followed by dehydration of the amide.

8. The method of either of claims 1 or 2 wherein R is a —COOR² group, and the step of converting a compound of formula (III) to a compound of formula (II) comprises the step of converting the —COOR² group to an amide followed by dehydration.

9. The method of either of claims 1 or 2 wherein R is a —CONR²R³ group, and the step of converting a compound of formula (III) to a compound of formula (II) comprises dehydrating the —CONR²R³ group to form the cyano group.

10. The method of either of claims 1 or 2 wherein R is a —NHR¹ group, and the step of converting a compound of formula (III) to a compound of formula (II) comprises hydrolysation to form a free amino group followed by diazotation of the free amino group and reaction with a cyanide source.

11. The method of either of claims 1 or 2 wherein R is a —CH₂NO₂ group, and the step of converting a compound of formula (III) to a compound of formula (II) comprises treating the compound of formula (III) with TMSI to form the cyano group.

12. The method of either of claims 1 or 2 wherein R is a —CH₂NH₂ group, and the step of converting a compound of formula (III) to a compound of formula (II) comprises oxidizing the compound of formula (III) in the presence of Copper(I)chloride to form the cyano group.

13. The method of either of claims 1 or 2 wherein R is a —CH₂Cl group, and the step of converting a compound of formula (III) to a compound of formula (II) comprises reacting the compound of formula (III) with AgNO₂ to form the corresponding —CH₂NO₂ group and treating the resulting compound with TMSI to form the cyano group.

14. The method of either of claims 1 or 2 wherein R is a —CH₂Br group, and the step of converting a compound of formula (III) to a compound of formula (II) comprises reacting the compound of formula (III) with AgNO₂ to form the corresponding —CH₂NO₂ group, and treating the resulting compound with TMSI to form the cyano group; or
treating the resulting compound with NH₃ to form the corresponding —CH₂NH₂ group and followed by an oxidation in the presence of Copper(I)chloride to form the cyano group.

15. The method of either of claims 1 or 2 wherein R is a —CH₃ group, and the step of converting a compound of formula (III) to a compound of formula (II) comprises treating the compound of formula (III) with a base and secondly with R⁹ONO₂, wherein R⁹ is a C₁₋₆-alkyl, to form the corresponding —CH₂NO₂ group, and treating the resulting compound with TMSI to form the cyano group.

16. The method of either of claims 1 or 2 wherein R is a —CH₂OH group, and the step of converting a compound of formula (III) to a compound of formula (II) comprises treating a compound of formula (III) with SOCl₂ or SOBr₂ to form the corresponding —CH₂Cl group or —CH₂Br group, followed by:
i) reacting the resulting compound with AgNO₂ to form the corresponding —CH₂NO₂ group, and treating the resulting compound with TMSI to form the cyano group; or
ii) treating the resulting compound with NH₃ to form the corresponding —CH₂NH₂ group, and oxidizing the resulting compound in the presence of Copper(I)chloride to form the cyano group.

17. The method of claim 3 wherein the cyanide source is selected from KCN, NaCN, Zn(CN)₂, CuCN, (R″)₄NCN wherein each R″ represents C₁₋₈-alkyl, or optionally two R″ together with the nitrogen form a ring structure, or combinations thereof.

18. The method of claim 4 wherein the cyanide source is selected from KCN, NaCN, Zn(CN)₂, CuCN, (R″)₄NCN wherein each R″ represents C₁₋₈-alkyl, or optionally two R″ together with the nitrogen form a ring structure, or combinations thereof.

19. The method of claim 10 wherein the cyanide source is selected from KCN, NaCN, Zn(CN)₂, CuCN, (R″)₄NCN wherein each R″ represents C₁₋₈-alkyl, or optionally two R″ together with the nitrogen form a ring structure, or combinations thereof.

20. The method of claim 3 wherein Zn²⁺ or Cu⁺ is added in substoichiometric amounts in combination with another cyanide source.

21. The method of claim 4 wherein Zn²⁺ or Cu⁺ is added in substoichiometric amounts in combination with another cyanide source.

22. The method of claim 10 wherein Zn²⁺ or Cu⁺ is added in substoichiometric amounts in combination with another cyanide source.

23. An antidepressant pharmaceutical composition comprising citalopram manufactured by the method of claim 1 or 2.
